(19) United States
(12) Patent Application Publication
(10) Pub. No.: US 2011/0008819 A1
(43) Pub. Date: Jan. 13, 2011

(54) Method of Differentiating Stem Cells

(76) Inventors: Hiram Chipperfield, Singapore (SG); Norris Ray Dunn, Singapore (SG)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/865,454

(22) PCT Filed: Jan. 23, 2009
(86) PCT No.: PCT/SG2009/000034

§ 371 Date: Jul. 30, 2010

(30) Foreign Application Priority Data
Jan. 31, 2008 (SG) 200800957-3

(51) Int. Cl. C12Q 1/02; C12N 5/074; C12N 5/073; C12N 5/0735

(52) U.S. Cl. 435/29; 435/375; 435/353; 435/354; 435/363; 435/366

(57) ABSTRACT

There is provided an improved efficient method for differentiating stem cells into pancreatic endoderm cells and pancreatic hormone expressing and secreting cells which express Pdx-1 and C-peptide. The invention further provides screening methods for detecting factors of interest that will affect the differentiation of the stem cells into pancreatic endoderm cells.

### FIELD OF THE INVENTION

The present invention relates to methods to promote the differentiation of stem cells. In particular, the present invention provides an improved method for the formation of pancreatic endoderm and/or pancreatic hormone secreting cells.

### BACKGROUND OF THE ART

Stem cells, with their capability for self-regeneration in vitro and their ability to produce differentiated cell types, may be useful for replacing the function of aging or failing cells in nearly any organ system. Cell-replacement therapy may be used to treat many illnesses such as cardiovascular diseases, autoimmune diseases, diabetes, osteoporosis, cancers and burns.

Insulin-dependent diabetes mellitus (IDDM) is a good example of a disease that could be cured or ameliorated through the use of stem cells. IDDM is a disease characterized by elevated blood glucose due to insufficient secretion of the hormone insulin by the pancreas. In the absence of this hormone, the body's cells are not able to absorb glucose from the blood stream causing an accumulation in the blood. Persons with diabetes are at risk for major complications, including diabetic ketoacidosis, end-stage renal disease, diabetic retinopathy and amputation.

While medications such as injectable insulin and oral hypoglycemics allow diabetics to live longer, diabetes remains the third major killer, leads to reduced quality of life and presents a significant burden to direct health care costs. Restoration of function with implants of functional insulin-secreting pancreatic beta cells is a promising treatment approach. However, the limiting factor in this approach is the availability of an islet source that is safe, reproducible, and abundant. Furthermore, currently available implants are linked to life-long immunosuppression which is expensive and not always effective. Other problems include the variability and low yield of islets obtained via dissociation, and the enzymatic and physical damage that may occur as a result of the isolation process. In addition, there are issues of immune rejection and current concerns with xenotransplantation using porcine islets.

A possible way to overcome these problems is to generate functional β cells from stem cells and maintain them in vitro for use in treating diabetes. Attempts to address these problems were made in WO2006/083782 and WO2007/127927. However, each one of these references suffers from one or more disadvantages as the methods provided are inefficient. Carrying out the methods of these references result in only a small population of the stem cells differentiating into functional β cells. They are thus unproductive. The prior art methods thus fail to consistently yield particular cell types or yield an exceedingly low percentage of a particular differentiated cell type. There is also a lot of research that is done to determine the best method for formation of functional β cells from stem cells. However, most of them conclude that the induction of insulin expression may require additional signals that are only present in vivo and that are not completely characterised.

For the forgoing reasons, there still remains a significant need to develop conditions for establishing stem cell lines that can be expanded to address the current clinical needs, while retaining the potential to differentiate into pancreatic endoderm cells and/or pancreatic hormone expressing and/or secreting cells.

### SUMMARY OF THE INVENTION

The present invention addresses the problems above and in particular provides new and/or improved methods to differentiate stem cells in vitro towards pancreatic endocrine cells, capable of secreting insulin in a well-regulated fashion. The present invention takes an alternative approach to the methods of the prior art in improving the efficiency of differentiating human embryonic stem cells towards pancreatic endocrine cells.

According to a first aspect, the present invention provides at least one method for differentiating stem cells into cells expressing C-peptide comprising the steps of:
(a) culturing stem cells in the presence of at least one extracellular matrix;
(b) culturing the stem cells obtained from step (a) in at least one first medium comprising at least BMP4 and Activin A to obtain definitive endoderm cells;
(c) culturing the cells obtained from step (b) in at least one second medium comprising at least Activin A;
(d) culturing the cells obtained from step (c) in at least one third medium comprising at least one fibroblast growth factor (FGF), retinoic acid (RA) and at least one inhibitor of hedgehog signalling to obtain cells expressing Pdx-1; and
(e) culturing the cells obtained from step (d) in at least one fourth medium comprising at least one inhibitor of notch signalling to differentiate cells expressing Pdx-1 to C-peptide producing cells.

In particular, the method according to the invention may be a two-dimensional (2D) method of differentiation.

In the method of the present invention, the culturing of the cells in the first medium comprising of at least BMP4 and Activin A (step b) may be for at least 3 days. The culturing of the cells in the second medium comprising Activin A (step c) may be in the range from 4 days to 7 days.

In the method of the present invention, the concentration of BMP4 may be in the range of 10-200 ng/ml. In particular, it may be in the range of 40-60 ng/ml and more in particular it may be about 50 ng/ml.

In the method of the present invention, the concentration of Activin A may be in the range of 10-200 ng/ml. In particular, in the range of 40-60 ng/ml and more in particular, it may be about 50 ng/ml. In the method of the present invention, FGF may be FGF2, FGF10 and/or FGF18, the inhibitor of hedgehog signalling may be SANT-1 and/or inhibitor of notch signalling may be Gamma Secretase Inhibitor X or DAPT. In particular, the inhibitor of notch signalling may be used at a concentration of at least about 10 μM. In the method of the present invention, nicotinamide may be added together with the inhibitor of notch signalling, before or after the addition of the inhibitor of notch signalling.

In the method of the present invention, the stem cells may be selected from any of embryonic stem cells, foetal stem cells, or adult stem cells. Further, the stem cells may be selected from any of human, mouse, primate or rat origin.

In the method of the present invention, the extracellular matrix may be selected from the group consisting of MATRIGEL™, growth factor-reduced MATRIGEL™, laminin, and fibronectin.

According to another aspect, the present invention provides at least one method of identifying at least one factor that modulates differentiation of stem cells into cells expressing markers characteristic of endoderm lineage comprising the steps of:

(a) culturing at least one cell population prepared according to the invention, in the presence of at least one factor to be tested; and
(b) comparing differentiation of the cells in the presence and absence of the factor, wherein at least one difference in the differentiation in the presence of the factor is indicative of identification of at least one factor that modulates the differentiation of the cells.

In particular, the markers characteristic of the endoderm lineage comprises at least Sox17 and FoxA2.

According to another aspect, the present invention provides at least one method of identifying at least one factor that modulates differentiation of stem cells into cells expressing Pdx-1 comprising the steps of:

(a) culturing at least one cell population prepared according to the invention, in the presence of at least one factor to be tested; and
(b) comparing differentiation of the cells in the presence and absence of the factor, wherein a difference in the presence of the factor is indicative of identification of at least one factor that modulates the differentiation of the cells.

According to another aspect, the present invention provides at least one method of identifying at least one factor that modulates differentiation of stem cells into cells expressing Pdx-1 and C-peptide comprising the steps of:

(a) culturing at least one cell population prepared according to the invention, in the presence of at least one factor to be tested; and
(b) comparing differentiation of the cells in the presence and absence of the factor, wherein a difference in the presence of the factor is indicative of identification of at least one factor that modulates the differentiation of the cells.

### BRIEF DESCRIPTION OF THE FIGURES

FIG. 1 shows a schematic representation of the method of the present invention. It comprises the steps of culturing the cells in the presence of fibronectin and then treating the cells with BMP4 and Activin A for at least 3 days. The cells are then treated with Activin A for at least 4 days before the cells are differentiated to cells of endoderm lineage. Pdx-1 expression is induced in these cells by treatment of the cells with Retinoic Acid (RA), at least one Fibroblast Growth Factor (FGF), and at least one inhibitor of Hedgehog signalling, for example SANT-1, for at least 6 days. The Pdx-1 expressing cells are then treated with an inhibitor of Notch Signalling in the presence or absence of Nicotinamide to further differentiate the cells to C-peptide and Pdx-1 producing cells.

FIG. 2A shows that Sox17 expression is induced the quickest when the stem cell cultures were pulsed with BMP4 compared to cells that were treated with only Activin A. Sox17 induction appears to be more sustained and slightly stronger with the 3 day BMP4 pulse compared to the 6 hr pulse. The highest expression of Sox17 appears to be on day 3 of differentiation when the cells were BMP4 pulsed for 3 days. It appears that in the cells treated with Activin A only, Sox17 does not start to be strongly expressed until day 6 indicating a relatively leisurely differentiation pace.

FIG. 2B shows that the addition of BMP4 does not appear to greatly affect FoxA2 expression which gradually increases up to day 10 in all conditions.

FIGS. 3A, B and C show the results of a limited time-course experiment performed using cell lines HES-3, ESI014, ESI017 and ESI035. In FIG. 3A, it appears that there is indeed a stronger induction of Sox17 when the cell cultures were pulsed with BMP4. There appears to be a significant increase in the expression of Sox17 on days 3, 6, 10 and 12 when the cells were treated with BMP4 and Activin A compared to only Activin A. In FIG. 3B, it appears that there is a significant increase in the expression of FoxA2 on day 10 of differentiation and a slight increase in expression of FoxA2 on day 6 and day 12 in cells treated with BMP4 and Activin A compared to only Activin A. In FIG. 3C, it appears that there is a significant decrease in the expression of Oct4 on days 3, 6, 10 and 12 of differentiation when the cells were treated with BMP4 and Activin A compared to only Activin A. Due to the lower resolution of this second time-course it was not possible to determine if Sox17 expression was also earlier as well as stronger. Treatment of the cells with knockout serum replacement (KOSR) was used as a negative control.

FIGS. 4A and 4B show the same field of view. Hoechst-stained cell nuclei are shown in FIG. 4A, while FIG. 4B shows cells stained for Pdx-1 protein. For the stained cells, fields of view were photographed at random across the well based on the DAPI channel. The number of nuclei and Pdx-1 expressing cells were then manually counted in a blinded manner. The main bias in the counting was that some fields of view had to be discarded as the cells were too dense to distinguish individual cells. The counts for each line were the compilation of at least 5 fields of view.

FIGS. 5A, B, C and D show the effect of initial differentiation conditions on day 18 of differentiation by measuring the Pdx-1 expression in four cell lines, ES1035, ES1049, ES1051 and HES-3 respectively. It appears that in all four cell lines there is a higher expression of Pdx-1 when the cells were treated with BMP-4 and Activin A for the first 3 days of differentiation. It also appears that the addition of FGF2 at different stages during the first 9 days of differentiation does not result in the same effect in all four cell lines. Therefore, the effect of the addition of FGF2 varies, dependent on the cell line used. There is thus no conclusion then can be derived with regard to the effect of FGF2 on the induction of expression of Pdx-1.

FIGS. 6A and B show the immunohistochemical results of two wells of a 96-well plate in a screening for effective maturation growth factors that bring about differentiation in cell lines ESI035, ESI049 and ESI051. The cells with C-peptide expression are shown as bright dots, some of which are highlighted with black arrows. These cells with C-peptide expression are prominent amidst the cells surrounding them which are mainly Pdx-1 expressing cells. The Pdx-1 expressing cells are shown as grey dots or masses. As can be seen, only a small number of cells are C-peptide positive. At most, there were approximately 30 C-peptide positive cells in each well. In most cases, there were less than 5 C-peptide positive cells in each well. These C-peptide positive cells were found independent of the growth factors added. Therefore, none of the added growth factors had any consistent effect on C-peptide expression or even Pdx-1 expression.

FIGS. 7A and B show the immunohistochemical results of two wells that were treated with 50 ng/ml Activin A and 50 ng/ml BMP4 for 3 days followed by treatment with 50 ng/ml Activin A for another 3 days. The cells were then cultured in a medium comprising 3 μM RA, 50 ng/ml FGF2, 1 μM SANT-1 and 10 mM Nicotinamide for 6 days which was followed by inhibition of Notch signalling in the cells by the addition of over 10 μM Gamma Secretase Inhibitor X from day 9-18 of the differentiation. As can be seen, there are a significant number of C-peptide positive cells when the cells were fixed at day 21 of culture. The cells expressing C-peptide can be seen as bright spots and some are further highlighted with black arrows.

FIG. 8 shows the results of an experiment to optimize retinoic acid concentration in the induction of Pdx-1 in stem cells. The results of 2 plates, plate 1 and plate 2 are shown. In both plates the highest percentage of Pdx-1 positive cells per well is observed when the concentration of RA used was about 3.0 to 6.3 μM.

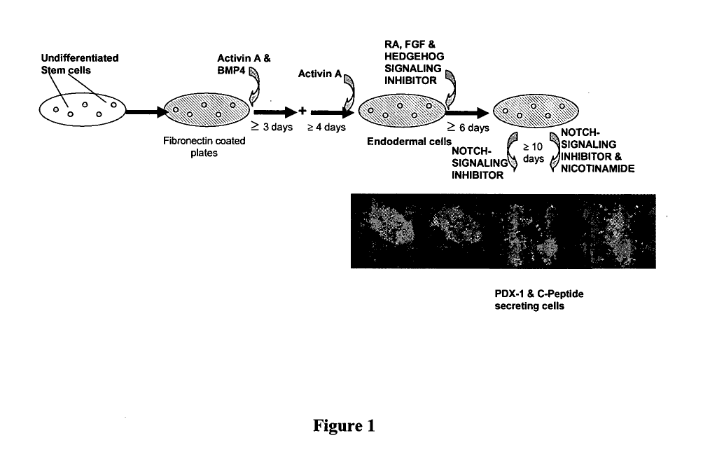

### DETAILED DESCRIPTION OF THE INVENTION

Bibliographic references mentioned in the present specification are for convenience listed in the form of a list of references and added at the end of the examples. The whole content of such bibliographic references is herein incorporated by reference.

### DEFINITIONS

For convenience, certain terms employed in the specification, examples and appended claims are collected here.

“Activin A” is a protein that plays a role in cell proliferation, differentiation, apoptosis, metabolism, homeostasis, immune response, wound repair, and endocrine function, and is produced in the gonads, pituitary gland, placenta and other organs. As with other members of the TGF-β superfamily, activins interact with two types of cell surface transmembrane receptors (Types I and II) which have intrinsic serine/threonine kinase activities in their cytoplasmic domains.

Bone Morphogenetic Protein 4 (“BMP4”) is a secreted polypeptide belonging to the BMP subgroup within the larger TGF-β superfamily of signaling cytokines. It, like other BMPs, is involved in bone and cartilage development, specifically tooth and limb development and fracture repair. During verteberate embryonic development, BMP4 is required for the establishment of the primary dorsal-ventral axis, the specification of primordial germ cells, and the initiation of mesoderm formation during gastrulation.

“C-peptide” is the abbreviation for “connecting peptide”. It is a stable peptide which is liberated when the proinsulin molecule is enzymatically cleaved into insulin and C-peptide in beta-cells. Both insulin and C-peptide are simultaneously released into the blood in response to increasing blood glucose levels. Therefore, an increase in C-peptide is directly proportional to an increase in insulin production. Even though they are produced at the same rate, C-peptide and insulin leave the body by different routes. Insulin is processed and eliminated mostly by the liver, while C-peptide is removed by the kidneys. Since the half-life of C-peptide is about 30 minutes compared to 5 minutes for insulin, normally there is about 5 times as much C-peptide in the bloodstream as insulin.

Fibroblast Growth Factors (“FGF”) are a family of secreted signaling molecules implicated in cell patterning, proliferation, differentiation, and survival in a wide range of tissues. There are currently 20 identified mammalian FGFs that are expressed throughout embryonic development and in the adult, and are implicated in a number of pathological conditions. A detailed description of the FGF family can be found in WO2004/011621.

“FGF2” is a member of the FGF family and is present in basement membranes and in the subendothelial extracellular matrix of blood vessels. It stays membrane-bound as long as there is no signal peptide. It has been hypothesized that, during both wound healing of normal tissues and tumor development, the action of heparan sulphate-degrading enzymes activates FGF2, thus mediating the formation of new blood vessels, a process known as angiogenesis.

“Hedgehog family members” belong to the hedgehog family of signaling molecules which mediate many important short- and long-range patterning processes during invertebrate and vertebrate development. Mammalian Hedgehog proteins include Sonic Hedgehog (Shh), Indian Hedgehog (lhh), and Desert Hedgehog (Dhh). Shh is expressed mainly in the epithelia in the tooth, hair, whisker, gut, bladder, limb, central nervous system, urethra, vas deferens, and lung, Dhh is found in Schwann and Sertoli cell precursors and Ihh is expressed in gut and cartilage.

“Notch Signaling Inhibitors” are inhibitors of the Notch signaling pathway which is a highly conserved cell signaling system present in most multicellular organisms. Notch signaling is one of the small numbers of signaling pathways frequently used during the development of metazoans to control different cell fate decisions. It mediates local cell-cell communications by using receptors and ligands that are present on the cell surface. “Oct-4” is a member of the POU-domain transcription factor family and is widely regarded as a hallmark of pluripotent stem cells. The relationship of Oct-4 to pluripotent stem cells is indicated by its tightly restricted expression to undifferentiated pluripotent stem cells. Upon differentiation to somatic lineages, the expression of Oct-4 disappears rapidly.

“Pdx-1” as used herein refers to a homeodomain transcription factor implicated in pancreas development also known as IPF-1, IDX-1 and Mody4. Pdx-1 confers the expression of pancreatic beta-cell-specific genes, such as genes encoding insulin, islet amyloid polypeptide, Glut2, and NR×6.1. Pdx-1 defines pancreatic cell lineage differentiation.

“SANT-1” is a Shh antagonist used. It acts as a potent antagonist of the Sonic Hedgehog (Shh) signaling pathway by binding directly to Smoothened (Smo), a distant relative of G protein-coupled receptors. SANT-1 equipotently inhibits the activities of both wild type and oncogenic Smo

“Shh” is a secreted morphogen and has been implicated in several embryonic developmental processes. It displays inductive, proliferative, neurotrophic, and neuroprotective properties. Shh signaling is required throughout embryonic development and is involved in the determination of cell fate and embryonic patterning during early vertebrate development. During the late stage of development, Shh is involved in the proper formation of a variety of tissues and organs. Shh also functions with other signaling molecules such as the fibroblast growth factors and bone morphogenetic protein to mediate developmental processes.

As used herein the term “animal” refers to mammals, preferably mammals such as humans. Likewise, a “patient” or “subject” to be treated by the method of the invention can mean either a human or non-human animal. The term “adult stem cell” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture.

The term “Cells expressing markers characteristic of the definitive endoderm lineage” as used herein refers to cells expressing at least one of the following markers: SOX-17, GATA-4, HNF-3 beta, GSC, Cetl, Nodal, FGF8, Brachyury, Mix-like homeobox protein (MixI1), FGF4 CD48, eomesodermin (EOMES), DKK4, FGF 17, GATA-6, CXCR4, C-Kit, CD99, or OTX2.

The term “cells expressing markers characteristic of the pancreatic endoderm lineage” as used herein refers to cells expressing at least one of the following markers: Pdx-I, HNF-(beta, PTF-1 alpha, HNF-6, or HB9.

The term “cells expressing markers characteristic of the pancreatic endocrine lineage” as used herein refers to cells expressing at least one of the following markers: NGN-3, NeuroD, Islet-1, Pdx-I, NKX6.1, Pax-4, Ngn-3, PTF-1 alpha or C-peptide. Cells expressing markers characteristic of the pancreatic endocrine lineage include pancreatic endocrine cells, pancreatic hormone expressing cells, and pancreatic hormone secreting cells, and cells of the β-cell lineage.

“Definitive endoderm” as used herein refers to cells which bear the characteristics of cells arising from the epiblast during gastrulation and which form the gastrointestinal tract and its derivatives. Definitive endoderm cells express the following markers: HNF-3 beta, GATA-4, SOX-17, Cerberus, goosecoid, C-Kit, CD99, and MixI1.

“Differentiation” in the present context means the formation of cells expressing markers known to be associated with cells that are more specialized and closer to becoming terminally differentiated cells incapable of further division or differentiation. For example, in a pancreatic context, differentiation can be seen as the progression of pancreatic endoderm maturing into functional hormone secreting endocrine cells.

The term “embryonic stem cell” is used to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970).

As used herein, the “lineage” of a cell defines the heredity of the cell, i.e., which cells it came from and what cells it can give rise to. The lineage of a cell places the cell within a hereditary scheme of development and differentiation. A lineage-specific marker refers to a characteristic specifically associated with the phenotype of cells of a lineage of interest and can be used to assess the differentiation of an uncommitted cell to the lineage of interest.

The term “β-cell lineage” refers to cells with positive gene expression for the transcription factor Pdx-1 and C-peptide.

The term “markers” as used herein are nucleic acid or polypeptide molecules that are differentially expressed in a cell of interest. In this context, differential expression means an increased level for a positive marker and a decreased level for a negative marker. The detectable level of the marker nucleic acid or polypeptide is sufficiently higher or lower in the cells of interest compared to other cells, such that the cell of interest can be identified and distinguished from other cells using any of a variety of methods known in the art.

“Matrigel™” (Collaborative Research, Inc., Bedford, Mass.) is a complex mixture of matrix and associated materials derived as an extract of murine basement membrane proteins, consisting predominantly of laminin, collagen IV, heparin sulfate proteoglycan, and nidogen and entactin, and was prepared from the EHS tumor (Kleinman et al, (1986) Biochemistry 25: 312-318).

The terms “pancreatic endocrine cell” or “pancreatic hormone expressing cell” as used herein refer to a cell capable of expressing at least one of the following hormones: insulin, glucagon, somatostatin, and pancreatic polypeptide.

The term “pancreatic hormone secreting cell” as used herein refers to a cell capable of secreting at least one of the following hormones: insulin, glucagon, somatostatin, and pancreatic polypeptide.

“Proliferation” indicates an increase in cell number.

“Stem cells” are undifferentiated cells defined by their ability at the single cell level to both self-renew and differentiate to produce progeny cells, including self-renewing progenitors, non-renewing progenitors, and terminally differentiated cells. Stem cells are also characterized by their ability to differentiate in vitro into functional cells of various cell lineages from multiple germ layers (endoderm, mesoderm and ectoderm), as well as to give rise to tissues of multiple germ layers following transplantation and to contribute substantially to most, if not all, tissues following injection into blastocysts.

Stem cells are classified by their developmental potential as: (1) totipotent, meaning able to give rise to all embryonic and extraembryonic cell types; (2) pluripotent, meaning able to give rise to all embryonic cell types; (3) multipotent, meaning able to give rise to a subset of cell lineages, but all within a particular tissue, organ, or physiological system (for example, hematopoietic stem cells (HSC) can produce progeny that include HSC (self-renewal), blood cell restricted oligopotent progenitors and all cell types and elements (e.g., platelets) that are normal components of the blood); (4) oligopotent, meaning able to give rise to a more restricted subset of cell lineages than multipotent stem cells; and (5) unipotent, meaning able to give rise to a single cell lineage (e.g. spermatogenic stem cells).

The term “tissue” refers to a group or layer of similarly specialized cells which together perform certain special functions.

The term “two-dimensional (2D)” refers to a 2D culture system for differentiating stem cells into pancreatic and/or pancreatic endocrine lineages. It involves culturing cells on flat plastic-ware and can result in a monolayer of adherent cells or layers of cells that may be several cells thick.

The term “three-dimensional (3D)” refers to a 3D culture system that resembles three dimensional environments that normal cells in vivo experience where they are completely surrounded by other cells, membranes, fibrous layers and adhesion proteins. It involves culturing the cells in three-dimensional scaffolds that reflects normal cell morphology and behaviour.

A person skilled in the art will appreciate that the present invention may be practised without undue experimentation according to the method given herein. The methods, techniques and chemicals are as described in the references given or from protocols in standard biotechnology and molecular biology text books.

In one aspect of the present invention, there is provided a method which is optimised for differentiation of stem cells into cells expressing C-peptide and/or Pdx-1. The method comprising the steps of:
(a) culturing stem cells in the presence of at least one extracellular matrix;
(b) culturing the stem cells obtained from step (a) in at least one first medium comprising at least BMP4 and Activin A to obtain definitive endoderm cells;
(c) culturing the cells obtained from step (b) in at least one second medium comprising at least Activin A;
(d) culturing the cells obtained from step (c) in at least one third medium comprising at least one fibroblast growth factor (FGF), retinoic acid (RA) and at least one inhibitor of hedgehog signalling to obtain cells expressing Pdx-1; and
(e) culturing the cells obtained from step (d) in at least one fourth medium comprising at least one inhibitor of notch signalling to differentiate cells expressing Pdx-1 to C-peptide producing cells.

The stem cells may be of human, mouse, primate or rat origin. In particular, the stem cells may be of human origin.

The stem cells may be selected from any of embryonic stem cells, fetal stem cells, or adult stem cells. In particular, the stem cells may be of embryonic origin. More in particular, the stem cells may be human embryonic stem cells.

The undifferentiated stem cells may first be cultured in at least one medium comprising feeders. The feeders that may be suitable for use in the present invention are described in WO2004/011621 (the whole content of which is herein incorporated by reference). The stem cells may then be harvested. For example, the stem cells may be harvested by treatment with collagenase IV when they become confluent. In particular, the cells may be harvested on day 7 after passage according to the method described in WO2006/083782 (the whole content of which is herein incorporated by reference).

The harvested cells may also be passed through at least one cell strainer to retain the large colony fragments. The pore size of the cell strainers used may be about 100 μM, 70 μM, 40 μM and the like. In particular, the cell strainer may have a pore size of about 100 μM.

The large colony fragments may be cultured in the presence of an extracellular matrix which may be selected from a group consisting of MATRIGEL™, growth factor-reduced MATRIGEL™, laminin, and fibronectin. In particular, the large colony fragments may be cultured in the presence of laminin or fibronectin. More in particular, the stem cells may be cultured in the presence of fibronectin for at least 2 days substantially in the absence of feeders.

The stem cells may be differentiated into cells expressing markers characteristic of endoderm lineage by culturing the stem cells in the presence of BMP4 and Activin A. In particular, the BMP4 may be used at a concentration in a range of 10 to 200 ng/ml, 20-100 ng/ml, 30-70 ng/ml, or 40-60 ng/ml. More in particular, the BMP4 may be used at a concentration of about 50 ng/ml. The Activin A may be used at a concentration in a range of 10 to 200 ng/ml, 20-100 ng/ml, 30-70 ng/ml, or 40-60 ng/ml. More in particular, the Activin A may be used at a concentration of about 50 ng/ml

More in particular, the stem cells may be cultured in at least one first medium comprising BMP4 and Activin A at a concentration of about 50 ng/ml each for at least 3 days.

The stem cells may then be cultured in at least one second medium comprising Activin A for at least 4 days. In particular, the stem cells may be cultured in the second medium comprising Activin A for 4 to 7 days with medium changes every 2 to 3 days.

The cells may then be cultured in at least one third medium comprising at least one fibroblast growth factor (FGF), retinoic acid (RA) and at least one inhibitor of hedgehog signalling. Although any FGF family member may be used to practice the methods of this invention, in particular, FGF2, FGF10 or FGF18 may be used. It is anticipated that the various FGF family members may have differential efficacies in the claimed methods. Multiple FGF family members may be used during the differentiation methods described herein (e.g., two or more FGF family members may be used at a particular step during the differentiation of the stem cells). In particular, FGF may be used in differentiation of stem cells at a concentration 5-100 ng/ml. More in particular, FGF may be used in the differentiation of stem cells at a concentration of about 50 ng/ml.

The inhibitor of hedgehog signalling may be a Shh antagonist. In particular, the Shh antagonist may be selected from a group consisting of AY9944, Cyclopamine, V. californicum, Cyclopamine-KAAD, Jervine, Purmorphamine, Tomatidine with HCl and SANT-1. More in particular, the Shh antagonist may be SANT-1. For example, SANT-1 may be used in the differentiation of stem cells at a concentration of about 10 μM.

The stem cells may be cultured in the third medium comprising FGF at about 50 ng/ml, RA at about 3 μM and SANT-1 at about 1 μM for at least 6 days with medium changes every 2 to 3 days. In particular, the cells at this stage may express markers characteristic of the endoderm lineage. More in particular, the marker characteristic of the pancreatic endoderm lineage may be Pdx-1.

The cells may be maintained in the third medium comprising FGF at about 50 ng/ml and SANT-1 at about 1 μM with medium changes every 2 to 3 days.

Pdx-1 positive cells may be treated with at least one inhibitor of notch signalling (Notch inhibitor) for at least 10 days. Treatment with the Notch inhibitor results in increase in the population of Pdx-1 positive cells differentiating to C-peptide producing cells compared to the population of Pdx-1 positive cells differentiating to C-peptide producing cells that may not be treated with the notch inhibitor. In particular the notch inhibitors may be gamma secretase inhibitor X and/or DAPT. For example, the notch inhibitor may be used at a concentration of at least 10 μM. In particular, nicotinamide may be added together with the notch inhibitor, before or after the notch inhibitor.

The Pdx-1 positive cells may be cultured in at least one fourth medium comprising gamma secretase inhibitor X at a concentration of at least 10 μM and nicotinamide at a concentration of about 10 mM for at least 10 days. In particular, the cells at this stage may express markers characteristic of pancreatic endocrine lineage. More in particular, the marker characteristic of the pancreatic endocrine lineage may be C-peptide. C-peptide level may be used as a measure of the presence of cells differentiated into pancreatic endocrine cells or beta-cells. Because man-made (synthetic) insulin does not have C-peptide, the presence of C-peptide in the differentiating cells of the present invention confirms that the C-peptide may be produced substantially by differentiated beta cells.

In another aspect of the present invention, there is provided at least one method of identifying at least one factor that modulates differentiation of stem cells into cells expressing markers characteristic of endoderm lineage comprising the steps of:

(a) culturing at least one cell population prepared according to the present invention, in the presence of at least one factor of interest to be tested; and
(b) comparing differentiation of the cells in the presence and absence of the factor, wherein at least one difference in the differentiation in the presence of the factor of interest is indicative of the identification of at least one factor that modulates the differentiation of the stem cells.

In particular, the factors of interest may be added in the first 6 days of differentiation. More in particular, the effect of the factor of interest on the differentiation of the stem cells into cells expressing markers characteristic of the endoderm lineage may be obtained by measuring at least one increase or decrease of the markers characteristic of the endoderm lineage. More in particular, the method for measurement may be RT-PCR, northern blot, in situ hybridization, Western Blot, immunohistochemical staining, ELISA and/or RIA.

For example, the method for measurement may be immunohistochemical staining. In particular, the cells may be stained for Oct4 and Sox17 for accessing endoderm differentiation.

In another aspect of the present invention, there is provided at least one method of identifying at least one factor that modulates differentiation of stem cells into cells expressing markers characteristic of pancreatic endoderm lineage comprising the steps of:

(a) culturing at least one cell population prepared according to the present invention, in the presence of at least one factor of interest to be tested; and
(b) comparing the differentiation of the cells in the presence and absence of the factor of interest, wherein at least one difference in the differentiation in the presence of the factor is indicative of the identification of at least one factor that modulates the differentiation of the stem cells.

In particular, factors may be added in days 2 to 12 of differentiation. More in particular, the effect of the factor on the differentiation of the stem cells into cells expressing markers characteristic of the pancreatic endoderm lineage may be obtained by measuring at least one increase or decrease of the markers characteristic of the pancreatic endoderm lineage. More in particular, the method for measurement may be RT-PCR, northern blot, in situ hybridization, Western Blot, immunohistochemical staining, ELISA and/or RIA. For example, the method for measurement may be immunohistochemical staining. In particular, the cells may be stained for FoxA2 and/or Pdx-1 for accessing pancreatic endoderm differentiation.

In another aspect of the present invention, there is provided at least one method of identifying at least one factor that modulates differentiation of stem cells into cells expressing markers characteristic of pancreatic endocrine lineage comprising the steps of:

(a) culturing at least one cell population prepared according to the present invention, in the presence of at least one factor of interest to be tested; and
(b) comparing the differentiation of the cells in the presence and absence of the factor of interest, wherein at least one difference in the differentiation in the presence of the factor is indicative of the identification of at least one factor that modulates the differentiation of the cells.

In particular, the factors may be added on day 10 and after and the cells may be stained for Pdx-1 and/or C-peptide for assessing beta cell differentiation. More in particular, the effect of the factor on the differentiation of the stem cells into cells expressing markers characteristic of the pancreatic endocrine lineage may be obtained by measuring at least one increase or decrease of the markers characteristic of the pancreatic endocrine lineage. For example, the method for measurement may be RT-PCR, northern blot, in situ hybridization, Western Blot, immunohistochemical staining, ELISA and/or RIA. In particular, the method for measurement may be immunohistochemical staining. In particular, the method for measurement may be immunocytochemistry. More in particular, the cells may be stained for Pdx-1 and/or C-peptide for assessing pancreatic endocrine differentiation.

Having now generally described the invention, the same will be more readily understood through reference to the following examples which are provided by way of illustration, and are not intended to be limiting of the present invention.

### EXAMPLES

Standard molecular biology techniques known in the art and not specifically described were generally followed as described in Sambrook and Russel, Molecular Cloning: A Laboratory Manual, Cold Springs Harbor Laboratory, New York (2001).

### Example 1

### Human Embryonic Stem Cell Culture

The human embryonic stem cell line HES-3 (http://www.nih.gov/) was obtained from ES Cell International Pte Ltd (Singapore) and, representative human embryonic stem cell lines ESI035, ES1049 and ESI051 were used to confirm the results obtained from the HES-3 cell line. These cells were cultured on Ortec feeders in hESC medium consisting of KO-DMEM, 20% Knockout Serum Replacement (KOSR), 1×NEAA and 1×L-glutamine. Ortec feeders were obtained from ES Cell International Pte Ltd (Singapore). Ortec feeders were expanded in DMEM, 10% FBS and 1×L-glutamine. Feeders were irradiated to arrest cell division a plated onto gelatine-coated plates at 2×10⁴/cm² overnight before the hES were plated on top of them. All culture reagents were obtained from Invitrogen. The cells were cultured at 37° C. in 5% CO₂ in a humidified tissue culture incubator.

When confluent (approximately 7 days after plating), human embryonic stem cells were treated with 1 mg/ml collagenase type IV (Invitrogen) for 10 min and then gently scraped off the surface using a cell scraper. Cells were passed through a 100 μM strainer and the large colony fragments retained. The large colony fragments were resuspended and re-plated on fibronectin coated multi-well tissue culture plastic plates in fresh hES culture medium. The cells were incubated in this medium for 2 days.

### Example 2

### Formation of Definitive Endoderm

The effects of Activin A with and without BMP4 on the expression of markers of definitive endoderm were examined. Activin A and BMP4 were added to a mix of cell lines, ESI017 and ES1035, of Example 1 after the cells had been washed twice with PBS. Four groups of cells were then cultured in four different conditions:

1) 50 ng/ml Activin A and 50 ng/ml BMP4 in a culture medium (2% B-27, 1×Glutamax, 1×NEAA, 1×β-mercaptoethanol in RPMI 1640 (RPMI)) for 3 days followed by treatment with Activin A (50 ng/ml) in RPMI.
2) 50 ng/ml Activin A and 50 ng/ml BMP4 in RPMI for about 6 hours followed by treatment with Activin A (50 ng/ml) in RPMI.
3) 50 ng/ml Activin A in RPMI.
4) 10% KOSR in RPMI (negative control).

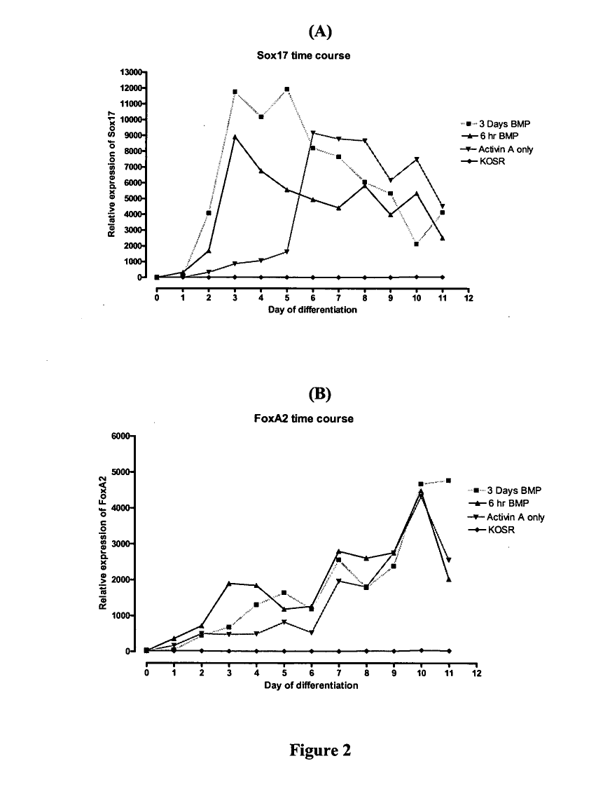

The cells were cultured continuously and a sample harvested daily. The medium was changed every 2-3 days. Real-time PCR was performed as described in Example 9. The results are shown in FIG. 2.

The combination of Activin A and BMP4 evoked a time-dependent increase in the expression of Sox17 mRNA in the mix of ESI017 and ESI035 cell lines for the first 3 days of differentiation (FIG. 2A). Sox17 expression was induced the quickest when the cultures were treated with BMP4 and Activin A compared to cells that were treated with only Activin A. Sox17 induction appeared to be more sustained and slightly stronger with the 3 day BMP4 pulse compared to the 6 hr pulse. The highest expression of Sox17 was on day 3 of differentiation when the cells were BMP4 pulsed for 3 days. The cells treated with Activin A only, had a weak expression of Sox17 until day 6 of the differentiation when the relative expression of Sox17 reached its maximum. The cells treated with Activin A only thus indicated a relatively leisurely differentiation pace. The addition of BMP4 did not appear to greatly affect FoxA2 expression which gradually increased up to day 10 in all conditions (FIG. 2B). The sustained and stronger expression of Sox17 within the first 3 days of differentiation suggest that Activin A in combination with BMP4 promotes a faster and more sustained differentiation of human embryonic stem cells to definitive endoderm.

### Example 3

In order to confirm the results of Example 2, a limited time-course experiment was performed using cells lines HES-3, ESI014, ESI017 and ESI035 from Example 1. Different groups of cells were then cultured in three different conditions:

1) 50 ng/ml Activin A and 50 ng/ml BMP4 in a culture medium (2% B-27, 1×Glutamax, 1×NEAA, 1×β-mercaptoethanol in RPMI 1640 (RPMI)) for 3 days followed by treatment with Activin A (50 ng/ml) in RPMI.
2) 50 ng/ml Activin A in RPMI.
3) 10% KOSR in RPMI (negative control).

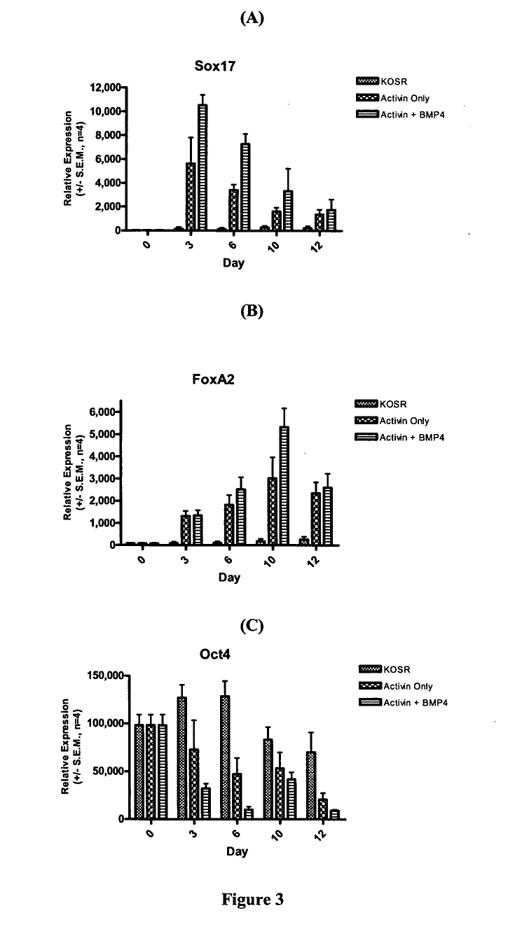

The medium was changed every 2-3 days. RNA was extracted according to Example 2 on days 0, 3, 6, 10 and 12 of differentiation. This follow-up experiment showed that there is indeed a stronger induction of Sox17 when the cultures were pulsed with BMP4 (FIG. 3). There was an increase in the expression of Sox17 on days 3, 6, 10 and 12 when the cells were treated with BMP4 and Activin A compared to only Activin A. There was also an increase in the expression of FoxA2 on day 10 of differentiation and a slight increase in expression of FoxA2 on day 6 and day 12 in cells treated with BMP4 and Activin A compared to only Activin A. There was a decrease in the expression of Oct4 on days 3, 6, 10 and 12 of differentiation when the cells were treated with BMP4 and Activin A compared to only Activin A. With the decrease in expression of Oct4, a marker of pluripotency, combined with an increase in the expression of definitive endoderm markers (Sox17 and FoxA2), these data suggest that the addition of BMP4 promotes the differentiation of human embryonic stem cells to definitive endoderm. Treatment of the cells with knockout serum replacement (KOSR) was used as a negative control.

### Example 4

### Formation of Pancreatic Endoderm

Growth factors known to induce the differentiation of human embryonic stem cells to pancreatic endoderm were added to cell cultures. In particular, Activin A, FGF2 also known as bFGF, retinoic acid (RA), and an inhibitor of hedgehog signalling such as SANT-1 which are known to induce the formation of pancreatic endoderm, were added to cell cultures of HES-3, ESI014, ESI017 and ESI035 from Example 1. These cell cultures were differentiated on parallel 24-well plates according to the following protocol.

The cells were first cultured for about 9 days in 50 ng/ml Activin A in a culture medium (2% B-27, 1×Glutamax, 1×NEAA, 1×β-mercaptoethanol in RPMI 1640 (RPMI)). RPMI with Activin A was then replaced with RPMI supplemented with 3 μM RA, 50 ng/ml FGF2 and 1 μM SANT-1. The medium was changed every 2-3 days. Cells were harvested on day 18 of differentiation and assayed by RT-PCR and immunohistochemistry for the expression of Pdx-1.

Table 1 below shows the results of the RT-PCR analysis, performed according to Example 9. Triplicate PCR wells were used for each sample. Each sample was normalised to β-actin expression and calculated using either the more rigorous standard curve method or the more practical ΔCt method. The results were then scaled by either making HES-3=1 or by using the equation expression=2^{20-ΔCt}. The results in the last three columns of Table 1 indicates that in all the cell lines tested, a small percentage of RA, FGF2 and SANT-1 treated cells expressed the Pdx-1 gene, a marker for pancreatic lineage.

TABLE 1
Comparison of counting Pdx-1+ cells and RT-PCR analysis.

| | | | Standard curve | | |
|---|---|---|---|---|---|
| | Pdx⁺ cells/ | Percentage | RT-PCR | Delta-CT RT- | Expression = |
| Line | Total cells | positive cells | (HES3 = 1) | PCR (HES3 = 1) | 2^{20-ΔCt} |
| HES3 | 798/2360 | 33.8% | 1 | 1 | 441 |
| ESI049 | 40/3461 | 1.2% | 0.0071 | 0.0038 | 1.66 |
| ESI051 | 278/4002 | 6.9% | 0.04 | 0.027 | 11.8 |
| ESI053 | 518/3944 | 13.1% | 0.03 | 0.018 | 8.1 |

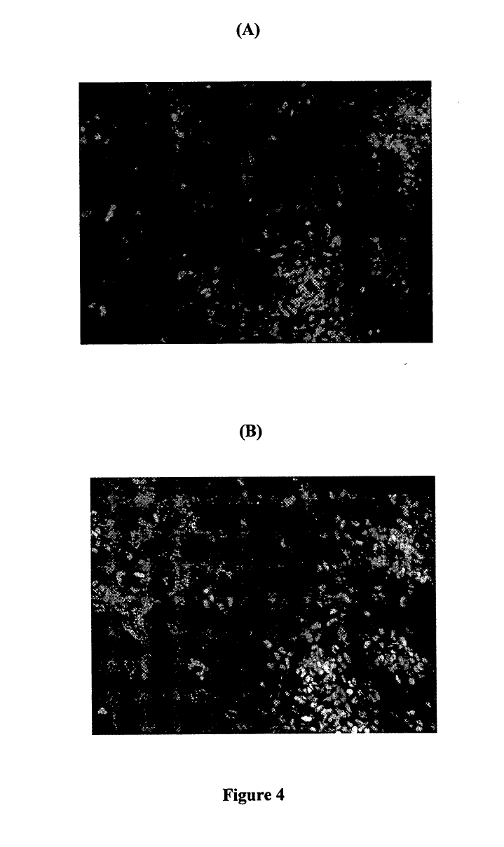

The results obtained from Immunohistochemistry performed according to Example 8 are shown in FIG. 4. FIG. 4(A) shows the staining of the nuclei and FIG. 4(B) the staining of the Pdx-1 expressing cells. For the stained cells, fields of view were photographed at random across the well based on the UV channel. The nuclei of the cells were stained with Hoechst (Invitrogen/GIBCO). The number of nuclei gave the total number of cells in each well. The number of nuclei and Pdx-1 expressing cells were then manually counted in a blinded manner. The main bias in the counting was that some fields of view had to be discarded as the cells were too dense to distinguish individual cells. The counts for each cell line were the compilation of at least five fields of view. The results of the counting are given in the first two columns of Table 1 under the heading “Pdx+ cells/Total cells”.

Analysis by immunohistochemistry revealed that protein expression for Pdx-1 also reflected the trends observed in the mRNA expression. That is, treatment of the cell lines with RA, FGF2 and SANT-1 did induce the expression of Pdx-1 in the cells but in some lines the percentage of cells that expressed the gene was low.

### Example 5

### Effect of early growth factors on cell lines ESI035, ES1049, ESI051 and HES-3

The effect of early differentiation factors was examined by differentiating cell lines ESI035, ESI049, ESI051 and HES-3 in 24-well plates in six varying cultures and harvesting them for analysis using immunohistochemistry and Quantitative RT-PCR analysis on day 18 and day 30 of differentiation. The culture medium (1×Glutamax, 1×NEAA, 1×β-mercaptoethanol in RPMI 1640 (RPMI)) was supplemented with other factors summarised in table 2 below. The medium was changed every 2-3 days.

TABLE 2
Summary of early growth factors added to cell lines ESI035, ESI049,
ESI051 and HES3 during 30 days of differentiation

| Day | KOSR | Short AA | A, A | AB, A | AB, AF | ABF, AF |
|---|---|---|---|---|---|---|
| −2 | 20% KOSR | 20% KOSR | 20% KOSR | 20% KOSR | 20% KOSR | 20% KOSR |
| −1 | 50 ng/ml FGF2 | 50 ng/ml FGF2 | 50 ng/ml FGF2 | 50 ng/ml FGF2 | 50 ng/ml FGF2 | 50 ng/ml FGF2 |
| 0 | 10% KOSR | 2% B27 | 2% B27 | 2% B27 | 2% B27 | 2% B27 |
| 1 | | 50 ng/ml Act A | 50 ng/ml Act A | 50 ng/ml Act A | 50 ng/ml Act A | 50 ng/ml Act A |
| 2 | | | | 50 ng/ml BMP4 | 50 ng/ml BMP4 | 50 ng/ml BMP4 |
| | | | | | | 50 ng/ml FGF2 |
| 3 | | | | 2% B27 | 2% B27 | 2% B27 |
| 4 | | | | 50 ng/ml ActA | 50 ng/ml ActA | 50 ng/ml ActA |
| 5 | | | | | 50 ng/ml FGF2 | 50 ng/ml FGF2 |
| 6 | | 2% B27 | | | | |
| 7 | | 3 μM RA | | | | |
| 8 | | 50 ng/ml FGF2 | | | | |
| 9 | | 1 μM SANT-1 | | | | |
| 10 | | | 2% B27 | 2% B27 | 2% B27 | 2% B27 |
| 11 | | | 3 μM RA | 3 μM RA | 3 μM RA | 3 μM RA |
| 12 | | 2% B-27 | 50 ng/ml FGF2 | 50 ng/ml FGF2 | 50 ng/ml FGF2 | 50 ng/ml FGF2 |
| 13 | | 50 ng/ml FGF2 | 1 μM SANT-1 | 1 μM SANT-1 | 1 μM SANT-1 | 1 μM SANT-1 |
| 14 | | 50 ng/ml EGF | | | | |
| 15 | | 1 μM SANT-1 | | | | |
| 16 | | | | | | |
| 17 | | | | | | |
| 18 | | | | | | |
| — | | | | | | |
| 30 | | | | | | |

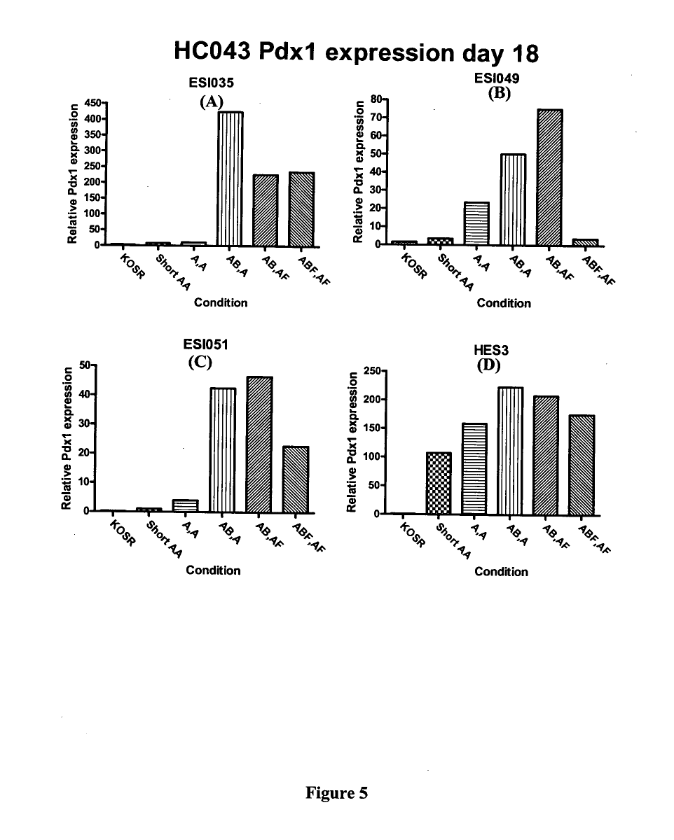

FIG. 5 shows the results of the RT-PCR analysis, performed according to Example 9. FIG. 5 shows the effect of different initial differentiation conditions according to Table 2 on Pdx-1 expression in the four cell lines, ES1035, ES1049, ES1051 and HES-3. All four cell lines showed a higher expression of Pdx-1 when the cells were treated with BMP4 and Activin A for the first 3 days of differentiation compared to the cells that were only treated with Activin A. The addition of FGF2 at different stages during the first 9 days of differentiation did not result in the same effect on Pdx-1 expression in all four cell lines. As the effect of FGF2 as an early growth factor varied depending on the cell line used, no conclusion was derived with regard to the effect of an early dose of FGF2 on the expression of Pdx-1.

Analysis of expression of Pdx-1 by immunohistochemistry revealed that protein expression for this gene also reflected the trends observed in mRNA expression. The number of cells which expressed Pdx-1 was estimated by visual inspection at day 18. The percentage of Pdx-1 positive cells was relatively higher in cells which were treated with Activin A and BMP4 in the first 3 days of differentiation (Table 3). Cell line ESI035 showed that cells treated with Activin A and BMP4 in the first 3 days, followed by treatment of cells with Activin A alone for the next 7 days (AB,A) resulted in the highest number of Pdx-1 positive cells, approximately 80%. These results corresponded to the results obtained from the mRNA expression thus validating the methods used.

TABLE 3
Percentages of Pdx-1+ cells as estimated by visual inspection.

| | KOSR | Short AA | A, A | AB, A | AB, AF | ABF, AF |
|---|---|---|---|---|---|---|
| HES3 | 0% | 20% | 50% | DEAD | DEAD | 90% |
| | | | | | | few cells |
| ESI035 | 0% | 3% | 5% | 80% | 70% | 60% |
| | | | | | | few cells |
| ESI049 | 0% | 0% | 4% | 20% | 15% | 10% |
| | | | | few cells | few cells | few cells |
| ESI051 | 0% | 0% | 0% | DEAD | DEAD | DEAD |

“DEAD” indicates few or no surviving cells.

Some plates were co-stained for Pdx-1 and C-peptide. No C-peptide positive cells were found in any of the wells. Also no Pdx-1 positive cells were found in the day 30 plate. Based on this, no RT-PCR analysis was performed for the day 30 samples. Insulin and glucagon were also assayed for the day 18 samples. No significant insulin or glucagon expression was detected and thus the results are not shown. There were also a number of wells with few or no surviving cells.

This was probably due in part at least, to a low number of cells initially plated in those wells as parallel wells contained healthy cells.

Taken together, these data suggest that the formation of Pdx-1 secreting pancreatic endoderm is further enhanced by culturing the cells in the medium comprising BMP4 (50 ng/ml) and Activin A (50 ng/ml) for the first 3 days of differentiation followed by treatment of the cells with Activin A (50 ng/ml) for the next 7 days and finally culturing the cells in a medium supplemented with 3 μM RA, 50 ng/ml FGF2 and 1 μM SANT-1. However, this was not sufficient to differentiate the cells to pancreatic endocrine C-peptide producing cells.

### Example 6

### Formation of Pancreatic Endocrine Cells

The effect of maturation growth factors was examined by differentiating cell lines ESI035, ESI049 and ESI051 from Example 1 in 96-well plates in two varying cultures and harvesting them for analysis using immunohistochemistry on day 18 of differentiation. The culture medium (1×Glutamax, 1×NEAA, 1×β-mercaptoethanol in RPMI 1640 (RPMI)) were supplemented with other factors summarised in Table 4 below. The notable changes to the differentiation protocol used here compared to that used in Example 4 were the shortened period of endoderm induction and the addition of nicotinamide from day 6 of differentiation. The medium was changed every 2-3 days.

TABLE 4
Summary of two experimental protocols used to screen for effective
maturation growth factors added to cell lines ESI035, ESI049 and ESI051
during 18 days of differentiation.

| Day | With BMP4 | Activin Only |
|---|---|---|
| −2 | 20% KOSR | 20% KOSR |
| −1 | 50 ng/ml FGF2 | 50 ng/ml FGF2 |
| 0 | 2% B27 | 2% B27 |
| 1 | 50 ng/ml Act A | 50 ng/ml Act A |
| 2 | 50 ng/ml BMP4 | |
| 3 | 2% B27 | |
| 4 | 50 ng/ml ActA | |
| 5 | | |
| 6 | 2% B27 | 2% B27 |
| 7 | 3 μM RA | 3 μM RA |
| 8 | 50 ng/ml FGF2 | 50 ng/ml |
| 9 | 1 μM SANT-1 | FGF2 |
| 10 | 20 mM Nico + | 1 μM SANT-1 |
| 11 | G.F. | 20 mM Nico + |
| | | G.F. |
| 12 | 2% B27 | 2% B27 |
| 13 | 50 ng/ml FGF2 | 50 ng/ml FGF2 |
| 14 | 1 μM SANT-1 | 1 μM SANT-1 |
| 15 | 20 mM Nico + | 20 mM Nico + |
| 16 | G.F. | G.F. |
| 17 | | |

A list of growth factors (G.F) were tested according to the protocol described in Table 4. These were namely, Laminin, TGF-alpha, FGF4, Activin A, TNF-alpha, FGF10, GDF-15, BMP4, FGF18, PDGF-AB, EGF, Noggin, HGF, M-CSF, LIF, NGF, PYY, Activin beta-B, GLP-1, DDL4, Exendin-4, Heparin, Insulin, VEGF, Betacellulin, Follistatin, TGF-beta and IGFII. There were added at two time points. Once with 3 μM RA, 50 ng/ml FGF2, 1 μM SANT-1, and 20 mM Nicotinamide at day 6 of differentiation and another with 50 ng/ml FGF2, 1 μM SANT-1, and 20 mM Nicotinamide at day 12 of differentiation.

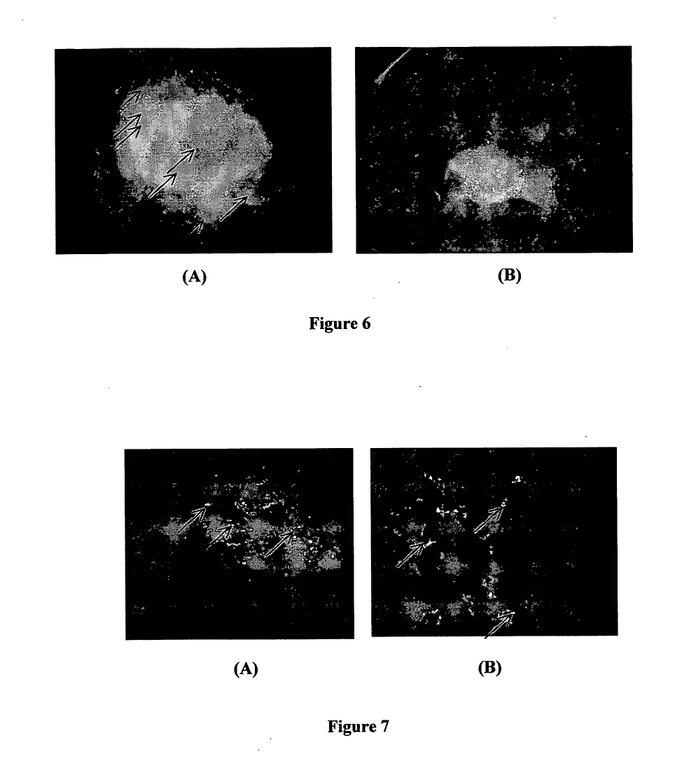

The results of the analysis using immunohistochemistry performed according to Example 8 revealed that the use of the growth factors resulted in the production of C-peptide positive cells (FIG. 6). The most C-peptide positive cells were found in the two wells shown in FIG. 6. Though these wells had the greatest number of C-peptide positive cells, they were still considered few and infrequent compared to the cells that did not produce C-peptide. At most, there were approximately 30 C-peptide positive cells in each well and in most of the wells, there were less than five C-peptide positive cells in each well. These C-peptide positive cells were found to be present independent of the growth factors added. All cell lines showed a higher frequency of C-peptide positive cells when initially cultured in a medium comprising BMP4. None of the added growth factors had any consistent effect on cell viability, C-peptide expression and Pdx-1 expression.

The results thus revealed that more Pdx-1 positive cells were found in the BMP4 treated plates for all the cell lines tested compared to the plates which were not BMP4 treated. The highest number and percentage of Pdx-1 positive cells were found in BMP4 treated cell line ESI049. There were less Pdx-1 positive cells found in BMP4 treated cell line ESI035 and even fewer were found in BMP4 treated cell line ESI051. The same trend across the different lines was evident in the Activin A only treatment group albeit with less overall Pdx-1 expression. Therefore, ESI049 seemed to be the cell line that was most sensitive to BMP4 treatment as it produced the highest number of Pdx-1 positive cells.

### Example 7

In order to increase the percentage of Pdx-1 and C-peptide producing cells, the cells from Example 1 were rinsed twice with PBS and then cultured in a culture medium (2% B-27, 1×Glutamax, 1×NEAA, 1×β-mercaptoethanol in RPMI 1640 (RPMI)) comprising 50 ng/ml Activin A and 50 ng/ml BMP4 for 3 days. The medium was then changed to a medium comprising 50 ng/ml Activin A in RPMI and incubated for 3 days. The cells were then cultured in a medium comprising 3 μM RA, 50 ng/ml FGF2, 1 μM SANT-1 and 10 mM Nicotinamide in RPMI for 6 days with the medium changed every 2-3 days. Notch signalling in the cells was then inhibited by the addition of Gamma Secretase Inhibitor X (DAPT) from day 9-18 of differentiation. Concentrations over 10 μM of either compound resulted in the production a significant number of C-peptide positive cells which were seen when the cells were fixed at day 21 of culture (FIG. 7).

### Example 8

### Immunohistochemistry

Plate-based immunohistochemistry was performed using standard techniques. Cells were fixed in 4% PFA in PBS for 30 min, washed with PBS and incubated in blocking solution consisting of 10% FCS and 0.1% Triton X-100 in PBS for 1 hr. Cells were incubated overnight at 4° C. with the primary antibody diluted in blocking solution. Examples of primary antibodies and their dilutions used include: guinea pig anti-Pdx-1 (1:10,000), rabbit anti-C-peptide (Linco, 1:1,000), goat anti-Sox17 (R & D systems, 1:100), goat anti-FoxA2 (R & D systems 1:100) and mouse anti-Oct4 (SantaCruz, 1:100). After incubation with the primary antibody the cells were washed with PBS and incubated with the secondary antibody diluted in blocking solution for about 2 hr at room temperature. The secondary antibodies were fluorescently conjugated antibodies from Jackson laboratories. In some cases double staining was performed; for example cells were stained using guinea pig anti-Pdx-1 and rabbit anti-C-peptide for primary antibodies followed by PE anti-guinea pig and Fitc anti-rabbit secondary antibodies. Finally the cells were incubated for about 5 min in Hoechst stain diluted in PBS to stain the nuclei, washed with PBS and visualised using an appropriate fluorescent microscope.

### Example 9

### RNA Isolation, cDNA Synthesis, and Quantitative PCR(RT-PCR)

Total RNA from hESC at various stages of differentiation was isolated using the Qiagen RNeasy kit (Invitrogen) according to the manufacturer's instructions. RNA was quantified by UV absorption. 1 to 5 μg of RNA was DNase I treated and converted to cDNA using M-MuLV reverse transcriptase (New England Biolabs) using random hexamer primers according to the manufacturer's instructions. Quantitative PCR(RT-PCR) was performed according to the manufacturer's instructions using a BioRad iCycler with approximately 50 ng cDNA per reaction containing 250 nM of each primer and 1×SYBR green master mix (Bio-Rad) and analyzed by Bio-RAD thermocycler.

The following conditions were used:

Quantitative PCR reaction:

| | |
|---|---|
| 2 × master mix | 15 μl |
| primers (each) | 100 nM |
| template | 25 ng |
| H2O | to 30 μl |

40 Cycles of:

30 s at 95° C.-denaturation
30 s at 55° C.-annealing
60 s at 72° C.-extension

### Analysis of Gene Expression

1) Cut and paste (or export) data to an excel spreadsheet.
2) Graph Ct (Y) vs. quantity (X) of standard curves. Convert X axis to log scale, Log(X). Get equation Y=slope * Log(X)+Y intercept. Under “options” include equation and R2 value on chart.
3) Determine input of unknown sample using the following equation (this can be prepared in the excel spreadsheet):
4) Input=10^{(Ctvalue-y intercept)/slope}
5) Repeat this procedure for the internal control of gene expression (GAPDH or β-actin)
6) Calculate the average input value of the three replicates for the gene and the internal control gene.
7) Calculate normalized expression of your gene using the following equation: normalized expression=Input value average of gene/Input value average of internal control gene.
8) Calculate the relative expression of your gene: Set one experimental condition as the comparison sample (untreated or time=0, for example). Relative expression=Normalized expression of unknown/normalized expression of comparison. Quality controls—

The slope of the curve you generate from the positive controls should be roughly equal to the slope in the standard curve chart below. If not, prepare fresh primer mix and standard curve reagents. To compare slopes, the trendlines must be generated in the same way. The slopes generated here use quantity in femtograms.

The Ct value of the unknown sample should be between the Ct values given by the positive controls. Otherwise, the results are outside the sensitive range of the assay.

Standard curves were generated by plotting the log(concentration in fg) of series of 100-fold dilutions of the target PCR amplicon (a range of 104 fg to 1 fg per reaction) versus the corresponding threshold Ct value. Normalized expression was determined by the following equation: Normalized expression=(input of target gene/input of actin control), where input is calculated as the inverse log of ((the threshold cycle (Ct value)−Y-intercept of standard curve)/slope of standard curve).

The specific primer pairs used are described in WO2006/083782 (the whole content of which is herein incorporated by reference).

### Example 10

### Screening protocol

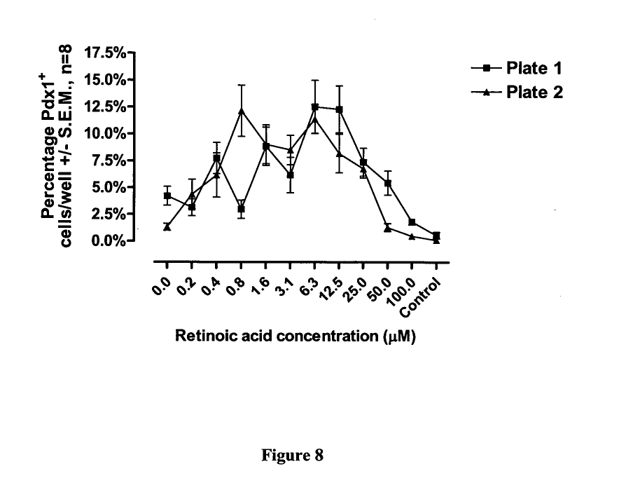

The stem cells were plated into black-walled, fibronectin coated, 96-well plates as given in Example 1. The stem cells were then differentiated according to Example 7. The factor of interest (RA) was added across a range of concentrations on days 6-12 of differentiation. The cells were immunostained according to Example 9 for Pdx-1 to assess pancreatic endoderm induction. Cell nuclei were visualized by staining with Hoechst. Multiple images of each well were automatically captured using a High Content Screening platform, in this example a Cellomics Arrayscan VTI. Using an automated spot finding algorithm the number of nuclei and Pdx-1 positive cells were automatically counted for each well for each concentration of RA used. FIG. 8 shows the results of 2 plates, plate 1 and plate 2. As can be seen, for both plates the highest percentage of Pdx-1 positive cells per well was observed when the concentration of RA used was about 3.0 to 6.3 μM.

### REFERENCES

Kleinman et al, (1986) Biochemistry. 25: 312-318.
U.S. Pat. No. 5,843,780
U.S. Pat. No. 5,945,577
U.S. Pat. No. 5,994,619
U.S. Pat. No. 6,200,806
U.S. Pat. No. 6,235,970
WO/2004/011621
WO/2007/127927

1. A method for differentiating stem cells into cells expressing C-peptide comprising the steps of:
(a) culturing stem cells in the presence of at least one extracellular matrix;
(b) culturing the stem cells obtained from step (a) in at least one first medium comprising at least BMP4 and Activin A to obtain definitive endoderm cells;
(c) culturing the cells obtained from step (b) in at least one second medium comprising at least Activin A;
(d) culturing the cells obtained from step (c) in at least one third medium comprising at least one fibroblast growth factor (FGF), retinoic acid (RA) and at least one inhibitor of hedgehog signalling to obtain cells expressing Pdx-1; and
(e) culturing the cells obtained from step (d) in at least one fourth medium comprising at least one inhibitor of notch signalling to differentiate cells expressing Pdx-1 to C-peptide producing cells.

2. The method according to claim 1, wherein the method is a two dimensional method of differentiation.

3. The method according to claim 1, wherein the culturing of the cells in the first medium in step b is for at least 3 days.

4. The method according to claim 1 wherein the culturing of the cells in the second medium in step c is in the range from 4 days to 7 days.

5. The method according to claim 1, wherein the concentration of BMP4 is in the range of 10-200 ng/ml.

6. The method according to claim 1, wherein the concentration of Activin A is in the range of 10-200 ng/ml.

7. The method according to claim 1, wherein FGF is FGF2, FGF10 and/or FGF18.

8. The method according to claim, wherein the inhibitor of hedgehog signalling is SANT-1.

9. The method according to claim 1, wherein the inhibitor of notch signalling is Gamma Secretase Inhibitor X and/or DAPT.

10. The method according to claim 1, wherein the inhibitor of notch signalling is used at a concentration of at least 10 μM.

11. The method according to claim 1, wherein nicotinamide is added together with, before or after the addition of inhibitor of notch signalling.

12. The method according to claim 1, wherein the stem cells are selected from any of embryonic stem cells, fetal stem cells, or adult stem cells.

13. The method according to claim 1, wherein the stem cells are selected from any of human, mouse, primate or rat origin.

14. The method according to claim 1, wherein the extracellular matrix is selected from the group consisting of MATRIGEL™, growth factor-reduced MATRIGEL™, laminin, and fibronectin.

15. A method of identifying at least one factor that modulates the differentiation of stem cells differentiated into cells expressing markers characteristic of the endoderm lineage comprising the steps of:
(a) culturing at least one cell population according to any one of the preceding claims, in the presence of at least one factor to be tested; and
(b) comparing the differentiation of the cells in the presence and absence of the factor, wherein at least one difference in the differentiation in the presence of the factor is indicative of the identification of at least one factor that modulates the differentiation of the cells.

16. The method according to claim 15, wherein the markers characteristic of the endoderm lineage comprise at least Sox17.

17. A method of identifying at least one factor that modulates the differentiation of stem cells differentiated into cells expressing Pdx-1 comprising the steps of:
(a) culturing at least one cell population according to claim 1, in the presence of at least one factor to be tested; and
(b) comparing the differentiation of the cells in the presence and absence of the factor, wherein at least one difference in the presence of the factor is indicative of the identification of at least one factor that modulates the differentiation of the cells.

18. A method of identifying at least one factor that modulates the differentiation of stem cells differentiated into cells expressing Pdx-1 and/or C-peptide comprising the steps of:
(a) culturing at least one cell population according to claim 1, in the presence of at least one factor to be tested; and
(b) comparing the differentiation of the cells in the presence and absence of the factor, wherein at least one difference in the presence of the factor is indicative of the identification of at least one factor that modulates the differentiation of the cells.
